(19) United States
(12) Patent Application Publication
(10) Pub. No.: US 2023/0104564 A1
(43) Pub. Date: Apr. 6, 2023

(54) CULTURE PROCESS FOR ENHANCING PROLIFERATION OF LACTIC ACID BACTERIA BY USING RED GINSENG

(71) Applicant: KOREA GINSENG CORP., Daejeon (KR)

(72) Inventors: Dong Bin RHIM, Daejeon (KR); Sang Kyu KIM, Daejeon (KR); Soon ki HONG, Daejeon (KR)

(73) Assignee: KOREA GINSENG CORP., Daejeon (KR)

(21) Appl. No.: 17/905,685

(22) PCT Filed: Mar. 5, 2021
(86) PCT No.: PCT/KR2021/002787

§ 371 Date: Sep. 6, 2022

(30) Foreign Application Priority Data
Mar. 6, 2020 (KR) 10-2020-0028491

(51) Int. Cl. C12N 1/04; C12N 1/20

(52) U.S. Cl. CPC C12N 1/04; C12N 1/20; C12R 2001/225

(57) ABSTRACT

Provided are a lactic acid bacteria culture medium comprising red ginseng-derived dietary fiber; and a method for culturing lactic acid bacteria by using same, wherein red ginseng-derived dietary fiber is used in a lactic acid bacteria culture medium so that the number of viable lactic acid bacteria can be increased and the culturing thereof can be improved, and the method uses the culture medium so as to improve culturing under the same conditions, and thus is advantageous in terms of cost.

### TECHNICAL FIELD

The present invention relates to a culture medium for lactic acid bacteria including a red ginseng raw material and a method for culturing lactic acid bacteria using the same.

### BACKGROUND ART

As ginseng (Panax ginseng C. A. Meyer) is a perennial herb belonging to the genus Panax of the Araliaceae family, which is one of the herbal medicines used in oriental medicine, it has a sweet and bitter taste, has a warm energy, and has herbal effects such as strengthening the vitality, protecting the spleen, and strengthening the lungs.

The main physiologically active substances of the ginseng include saponin, also called ginsenoside, an essential oil ingredient, polyacetylene, a phenol ingredient, glycoside, acidic peptide, and the like, and include additionally various ingredients such as vitamins, sugars, and minerals. The physiologically active efficacy of general ginseng has been reported to have an effect on the central nervous system, an effect of enhancing immune functions, an anticancer effect, and the like. In particular, about 40 types of ginsenoside have been discovered so far, and have been confirmed to exhibit excellent effects on the regulation of body functions, that is, normalization of physiological functions by widely affecting the endocrine system, the immune system, the metabolic system, and the like including the central nervous system. These ginsenosides have similar or opposite effects, and have been known to exhibit various effects by a specific ingredient alone or through interaction of several types thereof.

Meanwhile, dietary fiber is an ingredient known as a lot of fibers or cellulose contained in vegetables, fruits, and seaweed in foods, and refers to high molecular carbohydrates that are not digested by human digestive enzymes, but discharged out of the body. The fibers are known as a helpful ingredient in diet by making the stomach feel less hungry and retarding the absorption of food to filter cholesterol. The fibers are abundant in the tough part (cellulose) of vegetables, the brown pigment (lignin) of radish, pectin in fruits, and a sticky ingredient (alginic acid) of sea mustard and kelp, especially mushrooms.

Lactic acid bacteria are a generic term for bacteria that produce a lot of lactic acid as a metabolite and have been known to have physiological functions such as improvement of constipation, an increase in immunity, and the like by maintaining constant ratios of various intestinal microorganisms in the intestine and promoting intestinal movement. Since the lactic acid bacteria are usually vulnerable to temperature, acidity, pressure, etc., depending on a distribution process or a storage environment, the amount of lactic acid bacteria may not reach the recommended content of the Ministry of Food and Drug Safety. Therefore, research on a method for producing lactic acid bacteria to improve the stability of the lactic acid bacteria has been continuously conducted (Patent Document 1). In this context, in the incubation of lactic acid bacteria, since the viable cell count of lactic acid bacteria may vary greatly depending on the incubation conditions, studies on conditions capable of improving the culturability of lactic acid bacteria have also been continuously conducted.

While conducting various studies using red ginseng, the present inventors confirmed that in the case of using red ginseng-derived dietary fiber (fibre) excluding water-soluble ingredients of red ginseng in a culture medium for lactic acid bacteria, the viable cell count of lactic acid bacteria was increased and the culturability was greatly improved, and then completed the present invention.

### PRIOR ART DOCUMENT

### Patent Document

(Patent Document 1) Korean Patent Publication No. 10-2048690

### DISCLOSURE

### Technical Problem

It is an object of the present invention to provide a culture medium for lactic acid bacteria with improved culturability of lactic acid bacteria and a method for culturing lactic acid bacteria using the same.

### Technical Solution

An aspect of the present invention provides a culture medium for lactic acid bacteria including red ginseng-derived dietary fiber.

Another aspect of the present invention provides a method for culturing lactic acid bacteria including culturing lactic acid bacteria in a culture medium for lactic acid bacteria containing red ginseng-derived dietary fiber.

### Advantageous Effects

According to the present invention, since the viable cell count of lactic acid bacteria is increased by using red ginseng-derived dietary fiber in the culture medium for lactic acid bacteria, it is possible to improve the culturability. In addition, according to the method for culturing the lactic acid bacteria of the present invention, since the culturability is improved under the same conditions by using the culture medium, there is an advantage in terms of costs.

### DESCRIPTION OF DRAWINGS

FIG. 1 illustrates results of culturing Bifidobacterium lactis according to Examples and Comparative Examples.

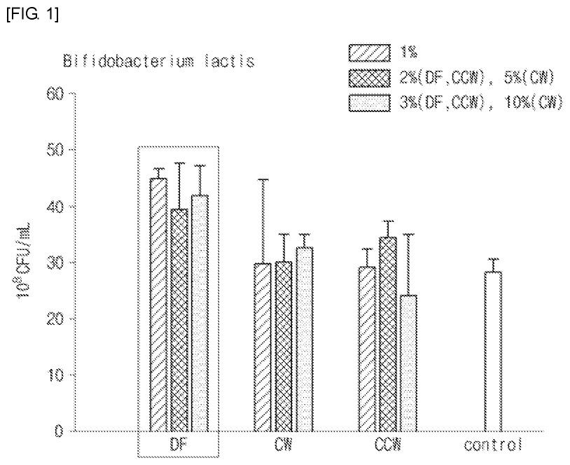

FIG. 2 illustrates results of culturing Bifidobacterium breve according to Examples and Comparative Examples.

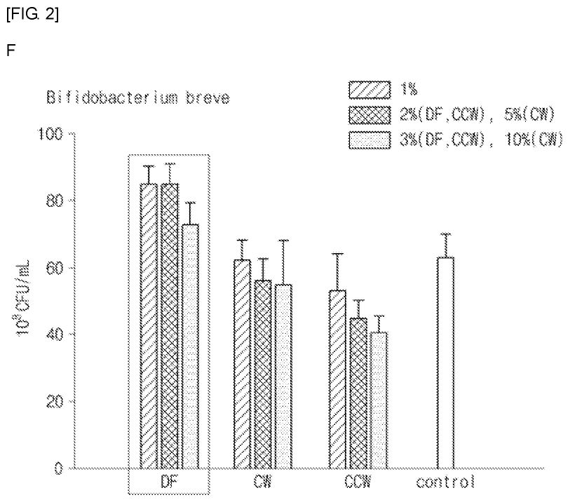

### BEST MODE

Hereinafter, the present invention will be described in more detail to help the understanding of the present invention.

Terms and words used in the present specification and claims should not be interpreted as being limited to typical or dictionary meanings, but should be interpreted as having meanings and concepts which comply with the technical spirit of the present disclosure, based on the principle that an inventor can appropriately define the concept of the term to describe his/her own invention in the best manner.

1. Culture Medium for Lactic Acid Bacteria

An aspect of the present invention provides a culture medium for lactic acid bacteria including red ginseng-derived dietary fiber.

In the present invention, ‘red ginseng’ means steamed ginseng (Panax ginseng). Specifically, the red ginseng may be manufactured by steaming the roots of ginseng with steam and then drying the steamed roots, and anything manufactured using ginseng as a raw material by conventional manufacturing methods used in the art may be used in the present invention. The red ginseng may have a reddish color as being manufactured through the same process as described above, and may be concentrated and contained with ingredients in ginseng. The amount and type of an active ingredient contained in the red ginseng, for example, saponin may be different from active ingredients contained in ginseng, and the red ginseng has less bitterness and stronger sweetness or better texture and easier storage than the ginseng. The ginseng may be used without limitation, such as cultivated or commercially available ginseng.

In the present invention, ‘dietary fiber’ refers to high molecular carbohydrates that are not digested by human digestive enzymes, but excreted out of the body, and may include, for example, pectin, gum, cellulose, lignin, and the like.

In the present invention, the ‘red ginseng-derived dietary fiber’ means dietary fiber obtained from red ginseng, and an obtaining method thereof is not limited. Specifically, the red ginseng-derived dietary fiber may be obtained and prepared from red ginseng by conventional methods, and may be obtained from a red ginseng raw material, an extract thereof, or a residue generated in the extraction process. For example, the red ginseng-derived dietary fiber may be extracted and obtained from the residue during the red ginseng extraction process, but when the dietary fiber is obtained from the residue generated in the process of preparing an extract of red ginseng used for various purposes, it is advantageous in terms of more efficiency and economy.

The red ginseng-derived dietary fiber may be extracted and obtained from the residue during the red ginseng extraction process using a centrifuge, a filter press, a belt press, a dryer, etc., but is not particularly limited thereto.

When the red ginseng-derived dietary fiber is extracted from the residue using the dryer, the drying process using the dryer may be performed at 80° C. or higher, 90° C. or higher, or 100° C. to 200° C., but is not particularly limited thereto.

The red ginseng extraction process may be performed using all conventionally known extraction methods, such as solvent extraction, ultrasonic extraction, filtration, and reflux extraction, and for example, solvent extraction or reflux extraction may be used. The extraction process may be repeated several times.

The red ginseng extraction process may be performed using water, an organic solvent, a supercritical fluid, a mixture thereof, or the like as an extraction solvent. The organic solvent may be alcohol, preferably C1-C4 lower alcohol, hexane (n-hexane), ether, glycerol, propylene glycol, butylene glycol, ethyl acetate, methyl acetate, dichloromethane, chloroform, ethyl acetate, benzene, mixed solvents, etc., for example, ethanol. For example, when a mixture of water and the organic solvent is used as the extraction solvent, the mixture of water and the organic solvent may be specifically a mixture of water and C1-C4 lower alcohol, more specifically, a mixture of water and ethanol.

The red ginseng extraction process may be performed at 0° C. to 100° C., specifically 40° C. to 90° C., more specifically 80° C. to 90° C., but is not limited thereto. The red ginseng extraction process may be performed for 6 hours to 36 hours, specifically 7 hours to 24 hours, more specifically 8 hours to 14 hours, but is not limited thereto. The extraction may be performed 1 to 8 times, specifically 1 to 6 times, and more specifically 1 to 5 times, but is not limited thereto.

In addition, the red ginseng-derived dietary fiber may further contain ingredients, such as ginsenosides such as Rb1, Rg3, Rg1, and Re, red ginseng polysaccharides, and the like, in addition to fibers. Accordingly, the red ginseng-derived dietary fiber of the present invention may exhibit various physiologically active functions by ginsenosides, red ginseng polysaccharides, etc. as well as exhibit functions by general fibers. In the present invention, the red ginseng-derived dietary fiber is used as a culture medium for lactic acid bacteria, thereby exhibiting excellent culturability.

The red ginseng-derived dietary fiber may be included in an amount of 0.001% (w/v) to 5% (w/v) in the culture medium for lactic acid bacteria of the present invention. Specifically, the red ginseng-derived dietary fiber may be included in an amount of 0.005% (w/v) to 4.5% (w/v), 0.01% (w/v) to 4%, 0.1% (w/v) to 3.5%, or 1% to 3%, but is not limited thereto. As the culture medium for lactic acid bacteria of the present invention includes the red ginseng-derived dietary fiber in the above content range, an effect of increasing the viable cell count by promoting the growth of lactic acid bacteria incubated in the medium is excellent. In particular, even when the content of red ginseng-derived dietary fiber is relatively low within the above range, the effect of promoting the growth of lactic acid bacteria is excellent, and thus, according to the purpose of incubation, the content of the dietary fiber may be appropriately selected and adjusted for use.

In the present invention, the ‘lactic acid bacteria’ are a generic term for bacteria that produce a lot of lactic acid as a metabolite, and may include, for example, the genus of Lactobacillus, Lactococcus, Enterococcus, Streptococcus, Leuconostoc, Pediococcus, Bifidobacterium, etc.

As an example, the lactic acid bacteria may include Lactobacillus acidophilus, Lactobacillus casei, Lactobacillus gasseri, Lactobacillus delbrueckii subsp. Bulgaricus, Lactobacillus helveticus, Lactobacillus fermentum, Lactobacillus paracasei, Lactobacillus plantarum, Lactobacillus reuteri, Lactobacillus rhamnosus, Lactobacillus salivarius, Lactobacillus delbrueckii subsp. lactis, Lactococcus lactis, Lactococcus lactis subsp. cremoris, Lactococcus lactis subsp. lactis biovar diacetylactis, Lactococcus lactis subsp. lactis, Enterococcus faecium, Enterococcus faecalis, Streptococcus thermophiles, Leuconostoc mesenteroides, Leuconostoc pseudomesenteroides, Bifidobacterium bifidum, Bifidobacterium breve, Bifidobacterium longum, Bifidobacterium animalis subsp. lactis, Bifidobacterium infantis, Bifidobacterium lactis, Bacillus coagulans, etc., but are not limited to the aforementioned lactic acid bacteria and include all lactic acid bacteria that can be used for foods.

The culture medium of the present invention is not particularly limited as long as being commonly used for culturing the lactic acid bacteria, and for example, MRS, BL, M17, NB, and BHI media, etc. may be used. As the culture medium of the present invention includes red ginseng-derived dietary fiber together with the ingredients contained in the medium commonly used for incubation of lactic acid bacteria, the viable cell count of lactic acid bacteria incubated using the medium may be significantly increased, thereby promoting the growth of lactic acid bacteria and improving the culturability.

The culture medium of the present invention may further include one or more selected from the group consisting of sugars, nitrogen sources, trace elements and carbonates as other ingredients necessary for culturing lactic acid bacteria.

The sugar ingredient may be mixed lactose, fructose, sucrose, glucose, maltose, trehalose, and the like.

The nitrogen source ingredient may be yeast extract, soy peptone, casein peptone, and the like.

The trace element may include potassium diphosphate, magnesium sulfate, manganese sulfate, calcium chloride, potassium citrate, sodium acetate, L-ascorbic acid, L-glutamic acid, L-cysteine hydrochloride, polysorbate 80, and the like.

The carbonate may be potassium carbonate, calcium carbonate, sodium carbonate, and the like.

2. Method for Culturing Lactic Acid Bacteria

Another aspect of the present invention provides a method for culturing lactic acid bacteria using the culture medium for lactic acid bacteria.

The method for culturing the lactic acid bacteria includes culturing the lactic acid bacteria in a culture medium containing red ginseng-derived dietary fiber.

For the ‘culture medium’, since the same contents as described above may be applied, a detailed description will be omitted.

First, the lactic acid bacteria are incubated in the culture medium.

The inoculation may be performed by inoculating the lactic acid bacteria at a concentration of 10⁷ CFU/ml to 10¹¹ CFU/ml, 5×10⁷ CFU/ml to 10¹⁰ CFU/ml or 10⁸ CFU/ml to 10⁹ CFU/ml in the culture medium, but is not limited thereto, and the concentration of the lactic acid bacteria to be inoculated may be appropriately selected according to a culture purpose, a culture time, etc. of the lactic acid bacteria.

Thereafter, the lactic acid bacteria are incubated.

The culturing step may be based on conventional conditions generally used for culturing the lactic acid bacteria in the art, and is not particularly limited.

For example, the incubation temperature condition may be a temperature of 30° C. to 40° C., 35° C. to 40° C., or 35° C. to 37° C. The lactic acid bacteria may be incubated under an incubation relative humidity condition, a relative humidity condition of 65% to 85%, 70% to 80%, or 72% to 78%. The culturing step may be culturing lactic acid bacteria under anaerobic conditions, for example, under conditions in which air is substituted with nitrogen gas or carbon dioxide gas. The incubation time for culturing the lactic acid bacteria may be 6 to 48 hours, 10 to 40 hours, or 15 to 24 hours.

When the lactic acid bacteria are incubated by the incubation method of the present invention, the growth of lactic acid bacteria is promoted to have an effect of dramatically increasing the viable cell count of lactic acid bacteria for the same time. Specifically, compared to a case of culturing lactic acid bacteria in a conventional culture medium for lactic acid bacteria, there is an advantage that the viable cell count may be increased by 16% to 59% in the case of culturing lactic acid bacteria by the incubation method of the present invention.

Hereinafter, the present invention will be described in detail by Examples and Comparative Examples.

However, the following Preparation Examples and Experimental Examples are just illustrative of the present invention, and the contents of the present invention are not limited to the following Preparation Examples and Experimental Examples.

### Example 1

Red ginseng was added with purified water, and extracted at a temperature of 87° C. for 24 hours to separate a red ginseng extract and a residue. To obtain red ginseng dietary fiber (DF), water was removed from the residue of red ginseng except for the red ginseng extract through a dehydration process and the residue was dried using a dryer at a temperature of 110° C. to obtain the red ginseng dietary fiber (DF).

A culture medium of the present invention was prepared by adding the red ginseng dietary fiber to an MRS medium commonly used for culturing lactic acid bacteria. Specifically, a medium (Example 1-1) added with the red ginseng dietary fiber at a concentration of 1% (w/v) of the total culture medium, a medium (Example 1-2) added with the red ginseng dietary fiber at a concentration of 2% (w/v), and a medium (Example 1-3) added with the red ginseng dietary fiber at a concentration of 3% (w/v) were prepared, and then used for incubation experiments for lactic acid bacteria.

### Comparative Examples 1 and 2

The culture media of Comparative Examples 1 and 2 were prepared by adding other ingredients derived from red ginseng to a conventional MRS medium without adding the red ginseng dietary fiber of Example 1. Specifically, culture media of Comparative Examples 1-1 to 1-3 were prepared by adding a ‘red ginseng-derived liquid (CW)’ obtained by collecting and then sterilizing a steamed ginseng effluent generated during a steaming process of ginseng to the MRS medium at concentrations of 1, 5 and 10% (w/v), respectively. Culture media of Comparative Examples 2-1 to 2-3 were prepared by adding a ‘red ginseng-derived concentrate (CCW)’ concentrated by centrifuging the steamed ginseng effluent to remove starch to the MRS medium at concentrations of 1, 2 and 3% (w/v), respectively.

### Experimental Example

Lactic acid bacteria were inoculated and incubated in the culture media of Examples and Comparative Examples, and viable cell counts were measured on MRS agar plates. As the lactic acid bacteria, Bifidobacterium lactis IDCC 4301 strain (LA 4301) and Bifidobacterium breve IDCC 4401 strain (BR 4401) were used. Each lactic acid bacteria strain was incubated up to 5×10⁹ CFU/ml, inoculated at 5% (v/v) into the culture media of Examples and Comparative Examples, and incubated for 40 hours under 37° C. temperature, 75% relative humidity, and anaerobic conditions, and then changes in viable cell count were measured. In a control group, a change in viable cell count was measured using an untreated group without adding red ginseng dietary fiber, and a rate of change in viable cell count compared to the control group was calculated as a percentage. The results were shown in Table 1 below.

TABLE 1

| | | Bifidobacterium lactis | | Bifidobacterium breve | |
|---|---|---|---|---|---|
| | | Viable cell | Rate of | Viable cell | Rate of |
| Classification | | count | change (%) | count | change (%) |
| Example 1-1(DF) | 1% (w/v) | 45.0 | 59 | 84.7 | 34 |
| Example 1-2(DF) | 2% (w/v) | 39.3 | 39 | 84.7 | 34 |
| Example 1-3(DF) | 3% (w/v) | 42.0 | 48 | 73.0 | 16 |
| Comparative Example | 1% (w/v) | 29.7 | 5 | 62.3 | −1 |
| 1-1(CW) | | | | | |
| Comparative Example | 5% (w/v) | 30.0 | 6 | 56.0 | −11 |
| 1-2(CW) | | | | | |
| Comparative Example | 10% (w/v) | 32.7 | 16 | 55.0 | −13 |
| 1-3(CW) | | | | | |
| Comparative Example | 1% (w/v) | 29.0 | 2 | 53.0 | −16 |
| 2-1(CCW) | | | | | |
| Comparative Example | 2% (w/v) | 34.3 | 21 | 45.0 | −29 |
| 2-2(CCW) | | | | | |
| Comparative Example | 3% (w/v) | 24.0 | −15 | 40.7 | −35 |
| 2-3(CCW) | | | | | |
| Control group | — | 28.3 | 0 | 63.0 | 0 |

As a result, in the culture media of Comparative Examples 1-1 to 1-3 and Comparative Examples 2-1 to 2-3 containing the red ginseng-derived liquid or the red ginseng-derived concentrate, the viable cell counts of lactic acid bacteria did not increase significantly or rather decreased compared to the control group. Specifically, in the case of culturing Bifidobacterium lactis, as the contents of the red ginseng-derived liquid or the red ginseng-derived concentrate increased in Comparative Examples, the viable cell count tended to slightly increase, but the values were not large. In the case of the medium of Comparative Example 2-3 containing 3% (w/v) of the red ginseng-derived concentrate, the viable cell count decreased by 15% and the effect of promoting the incubation of lactic acid bacteria was not largely exhibited. In addition, when the Bifidobacterium brev strain was incubated, it was confirmed that in the culture media of Comparative Examples, all viable cell counts were decreased to be unsuitable for culturing lactic acid bacteria.

On the other hand, in the culture media of Examples 1-1 to 1-3 containing the red ginseng dietary fiber of the present invention, both the case of culturing Bifidobacterium lactis and the case of culturing the Bifidobacterium brev strain, the viable cell counts of lactic acid bacteria were greatly increased. Specifically, the increase rate of the viable cell count was measured to be at least 16% and at most 59%, and when culturing the lactic acid bacteria in the culture medium of the present invention, it was confirmed that the culturability thereof was greatly improved. In addition, even in the case of using the culture medium (Example 1-1) containing the red ginseng dietary fiber in a small amount at 1% (w/v), the increase rate of the viable cell count was measured to be high. Accordingly, depending on the culture purpose, appropriate culture conditions may be set in various manners and even with a small amount of red ginseng dietary fiber, excellent effects may be achieved, so that it is expected that there is an advantage even in terms of costs.

1. A culture medium for lactic acid bacteria comprising red ginseng-derived dietary fiber.

2. The culture medium for lactic acid bacteria of claim 1, wherein the lactic acid bacteria comprises at least one selected from the group consisting of: Lactobacillus acidophilus, Lactobacillus casei, Lactobacillus gasseri, Lactobacillus delbrueckii subsp. Bulgaricus, Lactobacillus helveticus, Lactobacillus fermentum, Lactobacillus paracasei, Lactobacillus plantarum, Lactobacillus reuteri, Lactobacillus rhamnosus, Lactobacillus salivarius, Lactobacillus delbrueckii subsp. lactis, Lactococcus lactis, Lactococcus lactis subsp. cremoris, Lactococcus lactis subsp. lactis biovar diacetylactis, Lactococcus lactis subsp. lactis, Enterococcus faecium, Enterococcus faecalis, Streptococcus thermophiles, Leuconostoc mesenteroides, Leuconostoc pseudomesenteroides, Bifidobacterium bifidum, Bifidobacterium breve, Bifidobacterium longum, Bifidobacterium animalis subsp. lactis, Bifidobacterium infantis, Bifidobacterium lactis, and Bacillus coagulans.

3. The culture medium for lactic acid bacteria of claim 1, wherein the red ginseng-derived dietary fiber is obtained by extracting dietary fiber from a residue during a red ginseng extraction process.

4. The culture medium for lactic acid bacteria of claim 3, wherein the red ginseng-derived dietary fiber is extracted from the residue during the red ginseng extraction process using at least one selected from the group consisting of a centrifuge, a filter press, a belt press and a dryer.

5. A method for culturing lactic acid bacteria comprising inoculating and culturing lactic acid bacteria in a culture medium for lactic acid bacteria containing ginseng-derived dietary fiber.

6. The method for culturing lactic acid bacteria of claim 5, wherein the lactic acid bacteria comprises at least one selected from the group consisting of: Lactobacillus acidophilus, Lactobacillus casei, Lactobacillus gasseri, Lactobacillus delbrueckii subsp. Bulgaricus, Lactobacillus helveticus, Lactobacillus fermentum, Lactobacillus paracasei, Lactobacillus plantarum, Lactobacillus reuteri, Lactobacillus rhamnosus, Lactobacillus salivarius, Lactobacillus delbrueckii subsp. lactis, Lactococcus lactis, Lactococcus lactis subsp. cremoris, Lactococcus lactis subsp. lactis biovar diacetylactis, Lactococcus lactis subsp. lactis, Enterococcus faecium, Enterococcus faecalis, Streptococcus thermophiles, Leuconostoc mesenteroides, Leuconostoc pseudomesenteroides, Bifidobacterium bifidum, Bifidobacterium breve, Bifidobacterium longum, Bifidobacterium animalis subsp. lactis, Bifidobacterium infantis, Bifidobacterium lactis, and Bacillus coagulans.

7. The method for culturing lactic acid bacteria of claim 5, wherein the red ginseng-derived dietary fiber is obtained by extracting dietary fiber from a residue during a red ginseng extraction process.

8. The method for culturing lactic acid bacteria of claim 5, wherein the culturing is performed at a temperature of 35° C. to 40° C.

9. The method for culturing lactic acid bacteria of claim 5, wherein the culturing is performed under an anaerobic condition.

10. The method for culturing lactic acid bacteria of claim 5, wherein the culturing is performed for 6 to 48 hours.
